(19) United States
(12) Patent Application Publication
(10) Pub. No.: US 2021/0121460 A1
(43) Pub. Date: Apr. 29, 2021

(54) A TRIPLE PHARMACEUTICAL COMBINATION COMPRISING DABRAFENIB, TRAMETINIB AND AN ERK INHIBITOR

(71) Applicant: NOVARTIS AG, Basel (CH)

(72) Inventors: Matthew John Meyer, Framingham, MA (US); Youzhen Wang, Newton, MA (US)

(21) Appl. No.: 17/042,591

(22) PCT Filed: Mar. 29, 2019
(86) PCT No.: PCT/IB2019/052601

§ 371 Date: Sep. 28, 2020

Related U.S. Application Data
(60) Provisional application No. 62/650,744, filed on Mar. 30, 2018.

(51) Int. Cl. A61K 31/506; A61P 35/00; A61K 31/5025; A61K 9/00

(52) U.S. Cl. CPC A61K 31/506; A61K 9/0053; A61K 31/5025; A61P 35/00

(57) ABSTRACT

The present invention relates to a pharmaceutical combination comprising dabrafenib, trametinib and an Erk-inhibitor; pharmaceutical compositions comprising the same; and methods of using such combinations and compositions in the treatment or prevention of conditions in which MAPK pathway inhibition is beneficial, for example, in the treatment of cancers.

### FIELD OF THE INVENTION

The present invention relates to a pharmaceutical combination comprising dabrafenib, or a pharmaceutically acceptable salt thereof, trametinib or a pharmaceutically salt or solvate thereof, and an Erk inhibitor (ERKi) such as 4-(3-amino-6-((1S,3S,4S)-3-fluoro-4-hydroxycyclohexyl)pyrazin-2-yl)-N—((S)-1-(3-bromo-5-fluorophenyl)-2-(methylamino)ethyl)-2-fluorobenzamide (“Compound A” or “compound A”), or a pharmaceutically acceptable salt thereof; pharmaceutical compositions comprising the same; commercial packages comprising the same; and methods of using such combinations and compositions in the treatment or prevention of conditions in which MAPK pathway inhibition is beneficial, for example, in the treatment of cancers. The present invention also provides such combinations for use in the treatments of such conditions or cancers, including colorectal cancer (CRC) such as BRAF gain of function colorectal cancer.

### BACKGROUND OF THE INVENTION

The MAPK pathway is a key signaling cascade that drives cell proliferation, differentiation, and survival. Dysregulation of this pathway underlies many instances of tumorigenesis. Aberrant signaling or inappropriate activation of the MAPK pathway has been shown in multiple tumor types and can occur through several distinct mechanisms, including activating mutations in RAS and BRAF. The MAPK pathway is frequently mutated in human cancer with KRAS and BRAF mutations being among the most frequent (approximately 30%). RAS mutations, particularly gain of function mutations, have been detected in 9-30% of all cancers, with KRAS mutations having the highest prevalence (86%).

The extracellular signal-regulated kinases (ERKs) are one class of signaling kinases that are involved in conveying extracellular signals into cells and subcellular organelles. ERK1 and ERK2 are involved in regulating a wide range of activities and dysregulation of the ERK1/2 cascade is known to cause a variety of pathologies including neurodegenerative diseases, developmental diseases, diabetes and cancer. The role of ERK1/2 in cancer is of special interest because activating mutations upstream of ERK1/2 in its signaling cascade are believed to be responsible for more than half of all cancers. Moreover, excessive ERK1/2 activity was also found in cancers where the upstream components were not mutated, suggesting that ERK1/2 signaling plays a role in carcinogenesis even in cancers without mutational activations. The ERK pathway has also been shown to control tumor cell migration and invasion, and thus may be associated with metastasis.

The prognosis for patients suffering from certain cancers remains poor. Resistance to treatment occurs frequently and not all patients respond to available treatments. For example, the median survival for patients suffering from advanced colorectal cancer with BRAF mutation is less than 12 months. It is thus important to develop new therapies for patients suffering from cancer to achieve better clinical outcomes. Treatment options which are better tolerated and/or provide durable anti-tumor responses are also desired.

### SUMMARY OF THE INVENTION

The combinations of the present invention, dabrafenib, trametinib and an Erk-inhibitor such as Compound A, can be used as therapies for the treatment of diseases or disorders resulting from the aberrant activity of the MAPK pathway including, but not limited to, breast cancer, cholangiocarcinoma, salivary gland cancer, colorectal cancer, melanoma, non-small cell lung cancer, ovarian cancer and thyroid cancer. Combinations of dabrafenib, trametinib and an Erk-inhibitor such as Compound A are particularly useful in the treatment of colorectal cancer (CRC), including advanced or metastatic colorectal cancer, which is BRAF gain of function or BRAFV600E mutant.

The present invention provides a pharmaceutical combination comprising:

(a) N-(3-(5-(2-aminopyrimidin-4-yl)-2-(tert-butyl)thiazol-4-yl)-2-fluorophenyl)-2,6-difluorobenzenesulfonamide (dabrafenib), or a pharmaceutically acceptable salt thereof, having the structure:

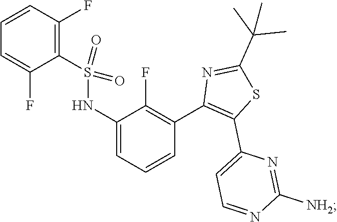 [SMILES: CC(C)(C)c1nc(-c2cccc(NS(=O)(=O)c3c(F)cccc3F)c2F)c(-c2ccnc(N)n2)s1]

(b) N-(3-(3-cyclopropyl-5-((2-fluoro-4-iodophenyl)amino)-6,8-dimethyl-2,4,7-trioxo-3,4,6,7-tetrahydropyrido[4,3-d]pyrimidin-1(2H)-yl)phenyl)acetamide (trametinib), or a pharmaceutically acceptable salt or solvate thereof, having the structure:

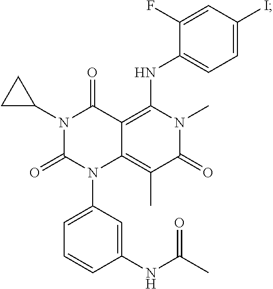 [SMILES: CC(=O)Nc1cccc(-n2c(=O)n(C3CC3)c(=O)c3c(Nc4ccc(I)cc4F)n(C)c(=O)c(C)c32)c1]

and
(c) 4-(3-amino-6-((1S,3S,4S)-3-fluoro-4-hydroxycyclohexyl)pyrazin-2-yl)-N—((S)-1-(3-bromo-5-fluorophenyl)-2-(methylamino)ethyl)-2-fluorobenzamide (Compound A), or a pharmaceutically acceptable salt thereof, having the structure:

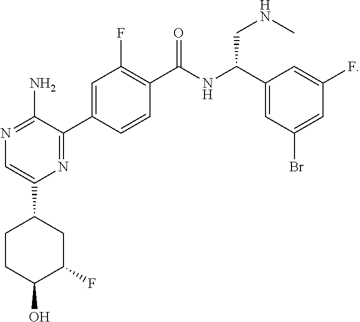 [SMILES: CNC[C@@H](NC(=O)c1ccc(-c2nc([C@H]3CC[C@H](O)[C@@H](F)C3)cnc2N)cc1F)c1cc(F)cc(Br)c1]

Pharmaceutical combinations of dabrafenib, or a pharmaceutically acceptable salt thereof, trametinib, or a pharmaceutically acceptable salt or solvate thereof, and Compound A, or a pharmaceutically acceptable salt thereof, will also be referred to herein as a “combination of the invention”.

There is provided a combination of the invention for use in the treatment of cancer, e.g for use in a cancer which is selected from breast cancer, cholangiocarcinoma, salivary gland cancer, colorectal cancer, melanoma, non-small cell lung cancer, ovarian cancer and thyroid cancer.

There is provided a pharmaceutical combination of dabrafenib, or a pharmaceutically acceptable salt thereof, trametinib, or a pharmaceutically acceptable salt or solvate thereof, and an Erk inhibitor for use in the treatment of cancer, e.g for use in a cancer which is selected from breast cancer, cholangiocarcinoma, salivary gland cancer, colorectal cancer, melanoma, non-small cell lung cancer, ovarian cancer and thyroid cancer. There is also provided a combination of combination of dabrafenib, or a pharmaceutically acceptable salt thereof, trametinib, or a pharmaceutically acceptable salt or solvate thereof, and an Erk inhibitor for use in the treatment of colorectal cancer (which includes advanced or metastsatic colorectal cancer) which is BRAF gain of function or BRAFV600E mutant.

Also provided herein is a combination of the invention for use in the treatment of colorectal cancer (which includes advanced or metastsatic colorectal cancer) which is BRAF gain of function or BRAFV600E mutant.

There is also provided an Erk-inhibitor such as Compound A, or a pharmaceutically acceptable salt thereof, for use in the treatment of cancer, e.g for use in a cancer which is selected from breast cancer, cholangiocarcinoma, salivary gland cancer, colorectal cancer, melanoma, non-small cell lung cancer, ovarian cancer and thyroid cancer, by co-administration with dabrafenib, or a pharmaceutically acceptable salt thereof, and trametinib, or pharmaceutically acceptable salt or solvate thereof.

There is also provided an Erk-inhibitor such as Compound A, or a pharmaceutically acceptable salt thereof, for use in colorectal cancer (which includes advanced or metastsatic colorectal cancer) which is BRAF gain of function CRC and in the treatment of BRAFV600E mutant colorectal cancer by co-administration with dabrafenib, or a pharmaceutically acceptable salt thereof, and trametinib, or pharmaceutically acceptable salt or solvate thereof.

In another embodiment of the combination of the invention, dabrafenib or a pharmaceutically acceptable salt thereof, trametinib, or a pharmaceutically acceptable salt thereof, and Compound A, or a pharmaceutically acceptable salt thereof, and are in the same formulation.

In another embodiment of the combination of the invention, dabrafenib or a pharmaceutically acceptable salt thereof, trametinib, or a pharmaceutically acceptable salt thereof, and Compound A or a pharmaceutically acceptable salt thereof, are in separate formulations.

In another embodiment, the combination of the invention is for simultaneous or sequential (in any order) administration.

In another embodiment, the present invention provides a method for treating cancer in a subject in need thereof comprising administering to the subject a therapeutically effective amount of the combination of the invention.

In a further embodiment of the method, the cancer is selected from breast cancer, cholangiocarcinoma, salivary gland cancer, colorectal cancer, melanoma, non-small cell lung cancer, ovarian cancer and thyroid cancer.

In a further embodiment, the combination of the invention provides for a use in the manufacture of a medicament for treating a cancer selected from breast cancer, cholangiocarcinoma, salivary gland cancer, colorectal cancer, melanoma, non-small cell lung cancer, ovarian cancer and thyroid cancer.

In a further embodiment, the present invention provides a combination of the invention for use in the manufacture of a medicament for treating a cancer selected from breast cancer, cholangiocarcinoma, salivary gland cancer, colorectal cancer, melanoma, non-small cell lung cancer, ovarian cancer and thyroid cancer.

In another embodiment there is provided a pharmaceutical composition or commercial package (e.g. a kit-of-parts) comprising the combination of the invention.

In a further embodiment, the pharmaceutical composition further comprises one or more pharmaceutically acceptable excipients.

### BRIEF DESCRIPTION OF THE DRAWINGS

FIG. 1: Anti-tumor effects of combination of dabrafenib+trametinib, dabrafenib+trametinib+Compound A and dabrafenib+trametinib+cetuximab.

FIG. 2: Body weight (BW) changes in the dabrafenib+trametinib, dabrafenib+trametinib+Compound A and dabrafenib+trametinib+cetuximab treated HCOX1329 model.

FIG. 3: Anti-tumor effects of combination of dabrafenib+trametinib and dabrafenib+trametinib+Compound A.

FIG. 4: Body weight changes in combination of dabrafenib+trametinib, dabrafenib+trametinib+Compound A and dabrafenib+trametinib+Cetiximab treated HCOX5421 model.

FIG. 5: Anti-tumor efficacy of Compound A, dabrafenib, trametinib, and combinations administered to athymic nude mice engrafted with the BRAF V600E HT29 human CRC xenograft model.

FIG. 6: Body weight changes associated with Compound A, dabrafenib, trametinib, and combinations administered to athymic nude mice engrafted with the BRAF V600E HT29 human CRC xenograft model. Final percent BW change was statistically similar (P>0.05) across all groups.

FIG. 7: DUSP6 transcript abundance in BRAF V600E HT29 CRC xenografts engrafted in nude mice administered the treatments indicated below the figure.

FIG. 8: DUSP6 transcript abundance in the dorsal skin of nude mice administered the treatments indicated below the figure.

FIG. 9: Anti-tumor efficacy of trametinib+dabrafenib, Compound A+trametinib, trametinib+dabrafenib+cetuximab, and Compound A+trametinib+dabrafenib administered to athymic nude mice engrafted with the BRAF V600E HT29 human CRC xenograft model.

FIG. 10: Body weight changes associated with Compound A, dabrafenib, trametinib, and combinations administered to athymic nude mice engrafted with the BRAF V600E HT29 human CRC xenograft model. Hash marks indicate instances where mice were removed from the study early.

### DESCRIPTION

The general terms used hereinbefore and hereinafter preferably have within the context of this disclosure the following meanings, unless otherwise indicated, where more general terms wherever used may, independently of each other, be replaced by more specific definitions or remain, thus defining more detailed embodiments of the invention:

“Dabrafenib” is N-(3-(5-(2-aminopyrimidin-4-yl)-2-(tert-butyl)thiazol-4-yl)-2-fluorophenyl)-2,6-difluorobenzenesulfonamide, a BRAF inhibitor (also known as: N-{3-[5-(2-Amino-4-pyrimidinyl)-2-(1,1-dimethylethyl)-1,3-thiazol-4-yl]-2-fluorophenyl}-2,6-difluorobenzenesulfonamide; Tafinlar®; & N-{3[5-(2-Amino-4-pyrimidinyl)-2-(1,1-dimethylethyl)-1,3-thiazol-4-yl]-2-fluorophenyl}-2,6 difluorobenzene sulfonamide, methanesulfonate salt).

“Trametinib” is N-(3-(3-cyclopropyl-5-((2-fluoro-4-iodophenyl)amino)-6,8-dimethyl-2,4,7-trioxo-3,4,6,7-tetrahydropyrido[4,3-d]pyrimidin-1(2H)-yl)phenyl)acetamide, a MEK inhibitor (also known as: N-{3-[3-cyclopropyl-5-(2-fluoro-4-iodo-phenylamino)6,8-dimethyl-2,4,7-trioxo-3,4,6,7-tetrahydro-2H-pyrido[4,3-d]pyrimidin-1-yl]phenyl} acetamide dimethyl sulfoxide solvate; Mekinist®).

“Cetuximab” is an epidermal growth factor receptor (EGFR) inhibitor used for the treatment of metastatic colorectal cancer, metastatic non-small cell lung cancer and head and neck cancer. Cetuximab is an epidermal growth factor receptor-targeted IgG1 monoclonal antibody that is approved for use in combination with irinotecan or as monotherapy in the treatment of metastatic CRC. Cetuximab is a chimeric (mouse/human) monoclonal antibody given by intravenous infusion.

“Compound A” is an inhibitor of extracellular signal-regulated kinases (ERK) 1/2. “Compound A” is 4-(3-amino-6-((1S,3S,4S)-3-fluoro-4-hydroxycyclohexyl)pyrazin-2-yl)-N—((S)-1-(3-bromo-5-fluorophenyl)-2-(methylamino)ethyl)-2-fluorobenzamide. A particularly preferred salt of Compound A is the hydrochloride salt thereof.

The term “subject” or “patient” as used herein is intended to include animals, which are capable of suffering from or afflicted with a cancer or any disorder involving, directly or indirectly, a cancer. Examples of subjects include mammals, e.g., humans, apes, monkeys, dogs, cows, horses, pigs, sheep, goats, cats, mice, rabbits, rats, and transgenic non-human animals. In an embodiment, the subject is a human, e.g., a human suffering from, at risk of suffering from, or potentially capable of suffering from cancers.

The term “treating” or “treatment” as used herein comprises a treatment relieving, reducing or alleviating at least one symptom in a subject or effecting a delay of progression of a disease. For example, treatment can be the diminishment of one or several symptoms of a disorder or complete eradication of a disorder, such as cancer. Within the meaning of the present disclosure, the term “treat” also denotes to arrest, delay the onset (i.e., the period prior to clinical manifestation of a disease) and/or reduce the risk of developing or worsening a disease.

The terms “comprising” and “including” are used herein in their open-ended and non-limiting sense unless otherwise noted.

The terms “a” and “an” and “the” and similar references in the context of describing the invention (especially in the context of the following claims) are to be construed to cover both the singular and the plural, unless otherwise indicated herein or clearly contradicted by context. Where the plural form is used for compounds, salts, and the like, this is taken to mean also a single compound, salt, or the like.

By “a combination” or “in combination with” or “co-administration with” and such like, it is not intended to imply that the therapy or the therapeutic agents must be physically mixed or administered at the same time and/or formulated for delivery together, although these methods of delivery are within the scope described herein. A therapeutic agent in these combinations can be administered concurrently with, prior to, or subsequent to, one or more other additional therapies or therapeutic agents. The therapeutic agents or therapeutic protocol can be administered in any order. In general, each agent will be administered at a dose and/or on a time schedule determined for that agent. It will further be appreciated that the additional therapeutic agent utilized in this combination may be administered together in a single composition or administered separately in different compositions. In general, it is expected that additional therapeutic agents utilized in combination be utilized at levels that do not exceed the levels at which they are utilized individually. In some embodiments, the levels utilized in combination will be lower than those utilized as single-agent therapeutics.

When describing a dosage or dose herein as ‘about’ a specified amount, the actual dosage or dose can vary by up to 10%, e.g. 5%, from the stated amount: this usage of ‘about’ recognizes that the precise amount in a given dose or dosage form may differ slightly from an intended amount for various reasons without materially affecting the in vivo effect of the administered compound. The skilled person will understand that where a dose or dosage of a therapeutic compound is quoted herein, that amount refers to the amount of the therapeutic compound in its free form or unsolvated form.

The phrase “therapeutically-effective amount” as used herein means that amount of a compound, material, or composition comprising a compound of the present invention which is effective for producing some desired therapeutic effect in at least a sub-population of cells in an animal (including a human) at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The combinations of the invention, dabrafenib, trametinib or compound A, is also intended to represent unlabeled forms as well as isotopically labeled forms of the compounds. Isotopically labeled compounds have one or more atoms replaced by an atom having a selected atomic mass or mass number. Examples of isotopes that can be incorporated into dabrafenib, trametinib and compound A include isotopes of hydrogen, carbon, nitrogen, oxygen, phosphorous, fluorine, and chlorine, such as ²H ³H, ¹¹C, ¹³C, ¹⁴C, ¹⁵N, ¹⁸F, ³¹P, ³²P, ³⁵S, ³⁶Cl, ¹²³I, ¹²⁴I, ¹²⁵I respectively. The invention includes isotopically labeled dabrafenib, trametinib and compound A, for example into which radioactive isotopes, such as ³H and ¹⁴C, or non-radioactive isotopes, such as ²H and ¹³C, are present. Isotopically labelled dabrafenib, trametinib and compound A are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, dabrafenib, trametinib or compound A labeled with ¹⁸F may be particularly desirable for PET or SPECT studies. Isotopically-labeled compounds of the invention can generally be prepared by conventional techniques known to those skilled in the art or by processes analogous to those described in the accompanying Examples using appropriate isotopically-labeled reagents.

Further, substitution with heavier isotopes, particularly deuterium (i.e., ²H or D) may afford certain therapeutic advantages resulting from greater metabolic stability, for example increased in vivo half-life or reduced dosage requirements or an improvement in therapeutic index. It is understood that deuterium in this context is regarded as a substituent of either dabrafenib, trametinib or compound A. The concentration of such a heavier isotope, specifically deuterium, may be defined by the isotopic enrichment factor. The term “isotopic enrichment factor” as used herein means the ratio between the isotopic abundance and the natural abundance of a specified isotope. If a substituent dabrafenib, trametinib or compound A is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

### DESCRIPTION OF PREFERRED EMBODIMENTS

Dabrafenib is an orally bioavailable small molecule with RAF inhibitory activity. Trametinib is an orally bioavailable small molecule with MEK inhibitory activity. Compound A is an orally bioavailable small molecule with ERK inhibitory activity. It is an inhibitor of extracellular signal-regulated kinases 1 and 2 (ERK 1/2).

In one embodiment, with respect to the pharmaceutical combination of the invention, is a pharmaceutical combination comprising N-(3-(5-(2-aminopyrimidin-4-yl)-2-(tert-butyl)thiazol-4-yl)-2-fluorophenyl)-2,6-difluorobenzenesulfonamide (dabrafenib), or a pharmaceutically acceptable salt thereof, N-(3-(3-cyclopropyl-5-((2-fluoro-4-iodophenyl)amino)-6,8-dimethyl-2,4,7-trioxo-3,4,6,7-tetrahydropyrido[4,3-d]pyrimidin-1(2H)-yl)phenyl)acetamide (trametinib), or pharmaceutically acceptable salt or solvate thereof, and 4-(3-amino-6-((1S,3S,4S)-3-fluoro-4-hydroxycyclohexyl)pyrazin-2-yl)-N—((S)-1-(3-bromo-5-fluorophenyl)-2-(methylamino)ethyl)-2-fluorobenzamide (compound A), or a pharmaceutically acceptable salt thereof.

In a further embodiment is a pharmaceutical combination wherein N-(3-(5-(2-aminopyrimidin-4-yl)-2-(tert-butyl)thiazol-4-yl)-2-fluorophenyl)-2,6-difluorobenzenesulfonamide (dabrafenib), or a pharmaceutically acceptable salt thereof, N-(3-(3-cyclopropyl-5-((2-fluoro-4-iodophenyl)amino)-6,8-dimethyl-2,4,7-trioxo-3,4,6,7-tetrahydropyrido[4,3-d]pyrimidin-1(2H)-yl)phenyl)acetamide (trametinib), or pharmaceutically acceptable salt or solvate thereof, and 4-(3-amino-6-((1S,3S,4S)-3-fluoro-4-hydroxycyclohexyl)pyrazin-2-yl)-N—((S)-1-(3-bromo-5-fluorophenyl)-2-(methylamino)ethyl)-2-fluorobenzamide (compound A), or a pharmaceutically acceptable salt thereof, are administered separately, simultaneously or sequentially, in any order.

In another embodiment, the pharmaceutical combination of the invention is for oral administration.

In a further embodiment, is a pharmaceutical combination wherein N-(3-(5-(2-aminopyrimidin-4-yl)-2-(tert-butyl)thiazol-4-yl)-2-fluorophenyl)-2,6-difluorobenzenesulfonamide (dabrafenib) is in an oral dosage form.

In a further embodiment, is a pharmaceutical combination wherein N-(3-(3-cyclopropyl-5-((2-fluoro-4-iodophenyl)amino)-6,8-dimethyl-2,4,7-trioxo-3,4,6,7-tetrahydropyrido[4,3-d]pyrimidin-1(2H)-yl)phenyl)acetamide (trametinib) is in an oral dosage form.

In a further embodiment, is a pharmaceutical combination wherein 4-(3-amino-6-((1S,3S,4S)-3-fluoro-4-hydroxycyclohexyl)pyrazin-2-yl)-N—((S)-1-(3-bromo-5-fluorophenyl)-2-(methylamino)ethyl)-2-fluorobenzamide (compound A) is in an oral dosage form.

In another embodiment is a pharmaceutical composition comprising the pharmaceutical combination according to any one of the preceding claims and at least one pharmaceutically acceptable carrier.

In another embodiment is a pharmaceutical combination of the invention for use in the treatment of cancer.

In a further embodiment, the cancer is selected from breast cancer, cholangiocarcinoma, salivary gland cancer, colorectal cancer, melanoma, non-small cell lung cancer, ovarian cancer and thyroid cancer.

In another embodiment, is the use of the pharmaceutical combination or the pharmaceutical composition for the manufacture of a medicament for the treatment of cancer.

In a further embodiment, the cancer is selected from breast cancer, cholangiocarcinoma, salivary gland cancer, olorectal cancer, melanoma, non-small cell lung cancer, ovarian cancer and thyroid cancer.

In another embodiment is a method of treating a cancer selected from breast cancer, cholangiocarcinoma, colorectal cancer, melanoma, non-small cell lung cancer, ovarian cancer and thyroid cancer comprising administrating to a patient in need thereof a pharmaceutical combination or the pharmaceutical composition of the invention.

In a further embodiment, N-(3-(5-(2-aminopyrimidin-4-yl)-2-(tert-butyl)thiazol-4-yl)-2-fluorophenyl)-2,6-difluorobenzenesulfonamide (dabrafenib) is administered orally at a dose of about 1, 2, 5, 10, 50, 100 or 150 mg per day. Thus, dabrafenib may be administered at a dose of from about 1 to about 150 mg per day, or at a dose which is selected from about 1, 2, 5, 10, 50, 100 and 150 mg daily in any method or use of the invention.

In a further embodiment, N-(3-(3-cyclopropyl-5-((2-fluoro-4-iodophenyl)amino)-6,8-dimethyl-2,4,7-trioxo-3,4,6,7-tetrahydropyrido[4,3-d]pyrimidin-1(2H)-yl)phenyl)acetamide (trametinib) dimethyl sulfoxide per day is administered orally at a dose of about 0.5635, 1.127 or 2.254 mg per day. Thus, trametinib may be administered at a dose of from about 0.5 to about 2 mg per day, or at dose which is selected from about 0.5, 1 and 2 mg daily in any method or use of the invention.

In a further embodiment, 4-(3-amino-6-((1S,3S,4S)-3-fluoro-4-hydroxycyclohexyl)pyrazin-2-yl)-N—((S)-1-(3-bromo-5-fluorophenyl)-2-(methylamino)ethyl)-2-fluorobenzamide (compound A) is administered orally at a dose of about 50, 75 or 100 mg per day. Thus, Compound A may be administered at a dose of from about 50 to about 100 mg per day, or at a dose which is selected from about 50, 75 and 100 mg per day in any method or use of the invention.

### Pharmacology and Utility

The MAPK pathway is frequently mutated in human cancer with KRAS and BRAF mutations being among the most frequent (approximately 30%). RAS mutations, particularly gain of function mutations, have been detected in 9-30% of all cancers, with KRAS mutations having the highest prevalence (86%), followed by NRAS (11%), and, infrequently, HRAS (3%) (Cox A D, Fesik S W, Kimmelman A C, et al (2014), Nat Rev Drug Discov. November; 13(11):828-51.). Although selective BRAF inhibitors (BRAFi), and to a lesser extent, MEK inhibitors (MEKi) have demonstrated good activity in BRAF-mutant tumors, currently no effective therapies exist for KRAS-mutant tumors (Cantwell-Dorris E R, O'Leary J J, Sheils O M (2011) Mol Cancer Ther. March; 10(3):385-94.).

The extracellular signal-regulated kinases (ERKs) are one class of signaling kinases that are involved in conveying extracellular signals into cells and subcellular organelles. ERK1 and 2 (ERK1/2) are kinases in the mitogen activated protein kinase (MAPK) pathway, and are also referred to as p42 and p44, respectively. ERK1 and ERK2 are present in relatively large quantities in cells (˜10⁷ molecules per cell), and are involved in regulating a wide range of activities. Indeed, dysregulation of the ERK1/2 cascade is known to cause a variety of pathologies including neurodegenerative diseases, developmental diseases, diabetes and cancer. Wortzel and Seger, Genes & Cancer, 2:195-209 (2011), published online 9 May 2011.

The role of ERK1/2 in cancer is of special interest because activating mutations upstream of ERK1/2 in its signaling cascade are believed to be responsible for more than half of all cancers. Moreover, excessive ERK1/2 activity was also found in cancers where the upstream components were not mutated, suggesting that ERK1/2 signaling plays a role in carcinogenesis even in cancers without mutational activations. The ERK pathway has also been shown to control tumor cell migration and invasion, and thus may be associated with metastasis. See A. von Thun, et al., ERK2 drives tumour cell migration in 3D microenvironments by suppressing expression of Rab17 and Liprin-β2, J. Cell Sciences, online publication date 10 Feb. 2012. In addition, it has been reported that silencing either ERK1 or ERK2 using shRNA killed melanoma cells in culture, and also made melanoma cells more sensitive to inhibitors of BRAF. J. Qin, et al., J. Translational Med. 10:15 (2012). It is also reported that inhibitors of ERK1 and 2 are effective on tumor cells resistant to MEK inhibitors, and that inhibition of MEK and ERK could simultaneously provide synergistic activity. Molec. Cancer Therapeutics, vol. 11, 1143 (May 2012).

Lung cancer is a common type of cancer that affects men and women around the globe. NSCLC is the most common type (roughly 85%) of lung cancer with approximately 70% of these patients presenting with advanced disease (Stage IIIB or Stage IV) at the time of diagnosis. About 30% of NSCLC tumors contain activating KRAS mutations, and these mutations are associated with resistance to EGFR tyrosine kinase inhibitors (TKIs) (Pao W, Wang T Y, Riely G J, et al (2005) PLoS Med; 2(1): e17). Activating KRAS mutations are also frequently found in melanoma (British J. Cancer 112, 217-26 (2015)), pancreatic cancer (Gastroenterology vol. 144(6), 1220-29 (2013)) and ovarian cancer (British J. Cancer 99(12), 2020-28 (2008)). BRAF mutations have been observed in up to 3% of NSCLC and have also been described as a resistance mechanism in EGFR mutation positive NSCLC.

Colorectal cancer (CRC), also known as bowel cancer and colon cancer, is the development of cancer from the colon or rectum. The prognosis for patients suffering from colorectal cancer is poor, specially for patients with BRAF mutation. The median survival is less than 12 months for this population. BRAF V600E mutations are present in 7 to 10% of CRC.

The following demonstrates the interplay of various MAPK pathway inhibitors. BRAF inhibition demonstrates robust single agent activity in melanoma. However, receptor tyrosine kinase reactivation occurs in BRAF gain-of-function colorectal cancer (CRC) which makes it less sensitive to BRAF inhibition. The response rate to the effect of a dual combination of MEK and BRAF inhibitors in BRAF gain-of-function CRC was found to be low, in the region of 12-29%. It was expected that adding an EGFR inhibitor such as cetuximab, which is already approved for the treatment of CRC, would enhance the response rate in this setting since adding an EGFR inhibitor to the combination of BRAF and MEK is susceptible to the upstream mutations in EGFR, RAS, RAF and MEK. However, the present inventors have surprisingly found that the combination of the invention, i.e. a triple combination of dabrafenib, trametinib and the ERK inhibitor, compound A, achieves a more durable anti-tumor response, e.g. compared to that obtained with a triple combination of dabrafenib, trametinib and an EGFR inhibitor.

The present invention therefore provides the combination of the present invention, dabrafenib, trametinib and an Erk-inhibitor such as Compound A, for use in therapies for the treatment of diseases or disorders resulting from the aberrant activity of the MAPK pathway including, but not limited to, breast cancer, cholangiocarcinoma, salivary gland cancer, colorectal cancer, melanoma, non-small cell lung cancer, ovarian cancer and thyroid cancer. The combination of the present invention is particularly useful in the treatment of colorectal cancer (which includes advanced or metastsatic colorectal cancer), e.g., particularly useful in the treatment of BRAF gain of function CRC and in the treatment of BRAFV600E mutant colorectal cancer.

### Pharmaceutical Compositions

In another aspect, the present invention provides pharmaceutically acceptable compositions which comprise a therapeutically-effective amount of dabrafenib, trametinib and compound A, formulated together with one or more pharmaceutically acceptable carriers (additives) and/or diluents. As described in detail below, the pharmaceutical compositions of the present invention may be specially formulated for administration in solid or liquid form, including those adapted for oral administration, for example, drenches (aqueous or non-aqueous solutions or suspensions), tablets, e.g., those targeted for buccal, sublingual, and systemic absorption, boluses, powders, granules, pastes for application to the tongue.

The phrase “pharmaceutically-acceptable carrier” as used herein means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

As set out above, certain embodiments of the present compounds may contain a basic functional group, such as amino or alkylamino, and are, thus, capable of forming pharmaceutically-acceptable salts with pharmaceutically-acceptable acids. The term “pharmaceutically-acceptable salts” in this respect, refers to the relatively non-toxic, inorganic and organic acid addition salts of compounds of the present invention. These salts can be prepared in situ in the administration vehicle or the dosage form manufacturing process, or by separately reacting a purified compound of the invention in its free base form with a suitable organic or inorganic acid, and isolating the salt thus formed during subsequent purification. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, for example, Berge et al. (1977) “Pharmaceutical Salts”, J. Pharm. Sci. 66:1-19).

The pharmaceutically acceptable salts of the subject compounds include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In other cases, the compounds of the present invention may contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of compounds of the present invention. These salts can likewise be prepared in situ in the administration vehicle or the dosage form manufacturing process, or by separately reacting the purified compound in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. (See, for example, Berge et al. (1977) “Pharmaceutical Salts”, J. Pharm. Sci. 66:1-19).

A particularly preferred salt of dabrafenib is the mesylate salt thereof. A particularly preferred solvate of trametinib is the dimethyl sulfoxide solvate thereof. A particularly preferred salt of compound A is the hydrochloride salt thereof.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

In certain embodiments, a formulation of the present invention comprises an excipient selected from the group consisting of cyclodextrins, celluloses, liposomes, micelle forming agents, e.g., bile acids, and polymeric carriers, e.g., polyesters and polyanhydrides; and a compound of the present invention. In certain embodiments, an aforementioned formulation renders orally bioavailable a compound of the present invention.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution, suspension or solid dispersion in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules, trouches and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions.

When the compounds of the present invention are administered as pharmaceuticals, to humans and animals, they can be given per se or as a pharmaceutical composition containing, for example, 0.1 to 99% (more preferably, 10 to 30%) of active ingredient in combination with a pharmaceutically acceptable carrier.

The compounds of the present invention and/or the pharmaceutical compositions of the present invention, are formulated into pharmaceutically-acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of the combination of the invention will be that amount of each compound which is the lowest dose effective to produce a therapeutic effect. Such an effective dose will generally depend upon the factors described above.

In another aspect, the present invention provides pharmaceutically acceptable compositions which comprise a therapeutically-effective amount of one or more of the subject compounds, as described above, formulated together with one or more pharmaceutically acceptable carriers (additives) and/or diluents.

### EXAMPLES

### Example 1

### Dabrafenib, Trametinib and Compound A

Dabrafenib is synthesized according to example 58a of WO2009/137391. Trametinib is synthesized according to example 4-1 of WO2005/121142. Compound A is synthesized according to example 184 of WO2015/066188. WO2005/121142, WO2009/137391 and WO2015/066188, are herein incorporated by reference in their entirety. The utility of a combination of Dabrafenib, trametinib and compound A described herein can be evidenced by testing in the following examples.

### Example 2

Effect of the Combination of Dabrafenib, Trametinib and Compound a on an In Vivo BRAF V600E CRC model HCOX1329

An in vivo antitumor efficacy study, employing mice engrafted with a BRAF V600E CRC (colorectal cancer) PDX (patient derived xenograft) model, was conducted to assess the therapeutic benefit of adding an ERK1/2 inhibitor such as compound A to the combination of the MEK1/2 inhibitor trametinib and the BRAF inhibitor dabrafenib. HCOX1329 was established by direct subcutaneous (sc) implantation of 4 million tumor cells into the right axillary region of 6-7 week old female nude (nu/nu) mice.

Mice were randomly assigned to treatment groups (summarized in the table below) 12 days post tumor fragment implantation with a tumor volume range between 153 to 325 mm³. Dabrafenib was formulated as solution in 0.5% HPMC (hydroxypropyl methylcellulose)+0.2% Tween 80 in pH8 DI water, 3 mg/mL. Trametinib was formulated as solution in 0.5% HPMC, 0.2% Tween 80 in pH8 DI Water, 0.03 mg/mL. Compound A was formulated as suspension in 0.5% HPC/0.5% Pluronic F127 in a pH 7.4 Phosphate Buffer, final pH adjusted to 4. Cetuximab was formulated as a solution in Phosphate buffered saline (PBS).

| Groups | Treatment | Dose | Schedule* | Number of mice |
|---|---|---|---|---|
| 1 | Vehicle | 0.5% HPMC + | QW, PO | 5 |
| | | 0.2% Tween 80 | | |
| | | in pH 8 DI Water | | |
| 2 | dabrafenib + | 30 mg/kg + | QD, PO + | 5 |
| | trametinib | 0.15 mg/kg | QD, PO | |
| 3 | dabrafenib + | 30 mg/kg + | QD, PO + | 5 |
| | trametinib + | 0.15 mg/kg + | QD, PO + | |
| | cetuximab | 20 mg/kg | 2QW, IP | |
| 4 | dabrafenib + | 30 mg/kg + | QD, PO + | 5 |
| | trametinib + | 0.15 mg/kg + | QD, PO + | |
| | compound A | 75 mg/kg | QD, PO | |

*QD or qd = once daily, PO or po = orally, 2QW or 2qw = twice per week, IP or ip = intraperitoneal injection

Animals were weighed at dosing day(s) and dose was body weight adjusted, dosing volume was 10 ml/kg. Tumor dimensions and body weights were collected at the time of randomization and twice weekly thereafter for the study duration. The following data were provided after each day of data collection: incidence of mortality, individual and group average body weights, and individual and group average tumor volume.

The % change in body weight was calculated as (BW_{current}−BWᵢₙᵢₜᵢₐₗ)/(BWᵢₙᵢₜᵢₐₗ)×100. Data are presented as percent body weight change from the day of treatment initiation.

Percent treatment/control (T/C) values were calculated using the following formulae:

% T/C=100×ΔT/ΔC if ΔT>0;

% Regression=100×ΔT/T₀ if ΔT<0;

where:
T=mean tumor volume of the drug-treated group on the final day of the study;
ΔT=mean tumor volume of the drug-treated group on the final day of the study—mean tumor volume of the drug-treated group on initial day of dosing;
T₀=mean tumor volume of the drug-treated group on the day of cohort;
C=mean tumor volume of the control group on the final day of the study; and
ΔC=mean tumor volume of the control group on the final day of the study—mean tumor volume of the control group on initial day of dosing.
Percent mice remaining on the study=6−number of mice reaching end point/6*100.

All data were expressed as mean±standard error of the mean (SEM). Delta tumor volume and percent body weight changes were used for statistical analysis. Between group comparisons were carried out using the One way ANOVA followed by a post hoc Tukey test. For all statistical evaluations, the level of significance was set at p<0.05. Significance compared to the vehicle control group is reported unless otherwise stated.

| Model | HCOX1329 |
|---|---|
| Treatment | T/C % or Reg % at day 25 |
| Vehicle | 100 |
| dabrafenib + trametinib | 26 |
| dabrafenib + trametinib + cetuximab | 29 |
| dabrafenib + trametinib + compound A | −47 |

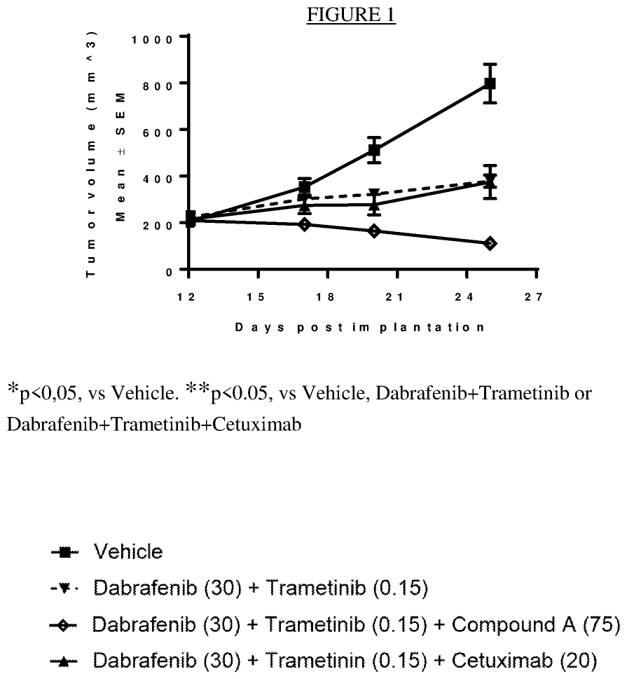

In HCOX1329 model (FIG. 1), the combination of dabrafenib (30 mg/kg, po qd) and trametinib (0.15 mg/kg, po qd) treatment produced a statistically significant anti-tumor effect with 22% T/C. The addition of ERK inhibitor compound A to the dabrafenib+trametinib combination led to tumor regression with 47% Reg, which is statistically significant as compared to the vehicle and combination of dabrafenib+trametinib (p<0.05). In contrast, there is no additional benefit with cetuximab to the dabrafenib+trametinib combination (29% T/C vs 26% T/C, p>0.05).

These data suggest that the combination of compound A+dabrafenib+trametinib_yields improved therapeutic benefit to patients with BRAF V600E CRC relative to the dabrafenib+trametinib double combination and also relative to the dabrafenib+trametinib+cetuximab triple combination. The agents in the triple combination of the present invention also benefit from being suitable for oral administration, and does not, e.g., have to be administered as an intravenous infusion.

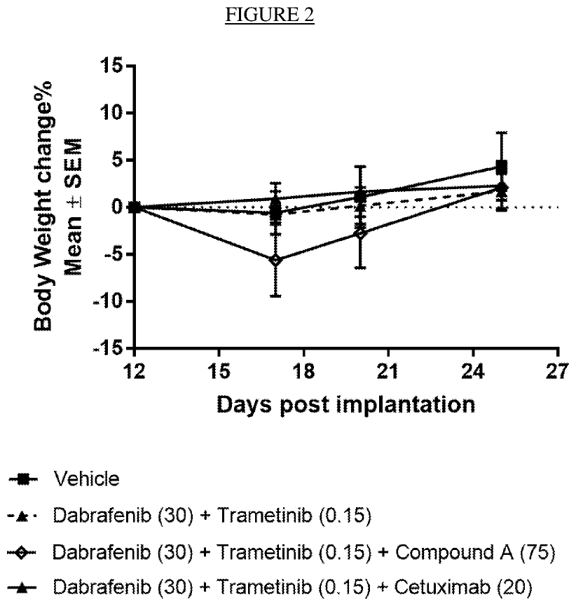

In addition, the mean body weight change for HCOX1329 is shown in FIG. 2. Treatment of mice with combinations of dabrafenib+trametinib, dabrafenib+trametinib+compound A and dabrafenib+trametinib+cetuximab exhibit body weight gains of 1.71%, 2.11% and 2.28%, respectively. No other signs of adverse events were observed in this study. These results indicate that the triple combination may be well tolerated. All animals survived throughout the study except for one animal in the dabrafenib+trametinib+compound A triple combination group.

### Example 3

### Effect of the Combination of Dabrafenib, Trametinib and Compound a on an In Vivo BRAF V600E CRC Model HCOX5421

An in vivo antitumor efficacy study, employing mice engrafted with a BRAF V600E CRC (colorectal cancer) PDX (patient derived xenograft) model HCOX5421, was conducted to assess the therapeutic benefit of adding an ERK1/2 inhibitor compound A to the combination of the MEK1/2 inhibitor trametinib and the BRAF inhibitor dabrafenib. HCOX5421 was established by direct subcutaneous (sc) implantation of a 50 mg tumor homogenate with 50% matrigel into the right axillary region of 6-7 week old female nude (nu/nu) mice.

Mice were randomly assigned to treatment groups (summarized in the table below) 11 days post tumor fragment implantation with a tumor volume range between 180 to 299 mm³. dabrafenib was formulated as a solution in 0.5% HPMC+0.2% Tween 80 in pH8 DI water, 3 mg/mL. trametinib was formulated as a solution in 0.5% HPMC, 0.2% Tween80 in pH8 DI Water, 0/03 mg/mL. Compound A was formulated as a suspension in 0.5% HPC/0.5% Pluronic F127 in a pH 7.4 Phosphate Buffer, final pH adjusted to 4. Cetuximab was formulated as a solution in PBS.

| Groups | Treatment | Dose | Schedule | Number of mice |
|---|---|---|---|---|
| 1 | Vehicle | 0.5% HPMC + | QW, PO | 5 |
| | | 0.2% Tween 80 | | |
| | | in pH 8 DI Water | | |
| 2 | dabrafenib + | 30 mg/kg + | QD, PO + | 5 |
| | trametinib | 0.3 mg/kg | QD, PO | |
| 3 | dabrafenib + | 30 mg/kg + | QD, PO + | 5 |
| | trametinib + | 0.3 mg/kg + | QD, PO + | |
| | cetuximab | 20 mg/kg | 2QW, IP | |
| 4 | dabrafenib + | 30 mg/kg + | QD, PO + | 7 |
| | trametinib + | 0.15 mg/kg + | QD, PO + | |
| | compound A | 75 mg/kg | QD, PO | |

*QD or qd = once daily, PO or po = orally, 2QW or 2qw = twice per week, IP or ip = intraperitoneal injection

Animals were weighed at dosing day(s) and dose was body weight adjusted, dosing volume was 10 ml/kg. Tumor dimensions and body weights were collected at the time of randomization and twice weekly thereafter for the study duration. The following data was provided after each day of data collection: incidence of mortality, individual and group average body weights, and individual and group average tumor volume.

The % change in body weight was calculated as (BW_{current}−BWᵢₙᵢₜᵢₐₗ)/(BWᵢₙᵢₜᵢₐₗ)×100. Data is presented as percent body weight change from the day of treatment initiation.

Percent treatment/control (T/C) values were calculated using the following formulae:

% T/C=100×ΔT/ΔC if ΔT>0;

% Regression=100×ΔT/T₀ if ΔT<0;

where:
T=mean tumor volume of the drug-treated group on the final day of the study;
ΔT=mean tumor volume of the drug-treated group on the final day of the study—mean tumor volume of the drug-treated group on initial day of dosing;
T₀=mean tumor volume of the drug-treated group on the day of cohort;
C=mean tumor volume of the control group on the final day of the study; and
ΔC=mean tumor volume of the control group on the final day of the study−mean tumor volume of the control group on initial day of dosing.

Percent mice remaining on the study=6−number of mice reaching end point/6*100.

All data were expressed as mean±standard error of the mean (SEM). Delta tumor volume and percent body weight changes were used for statistical analysis. Between group comparisons were carried out using the One way ANOVA followed by a post hoc Tukey test. For all statistical evaluations, the level of significance was set at p<0.05. Significance compared to the vehicle control group is reported unless otherwise stated.

| Model | HCOX5421 |
|---|---|
| Treatment | T/C % at day 11 |
| Vehicle | 100 |
| dabrafenib + trametinib | 41 |
| dabrafenib + trametinib + cetuximab | 29 |
| dabrafenib + trametinib + compound A | 20 |

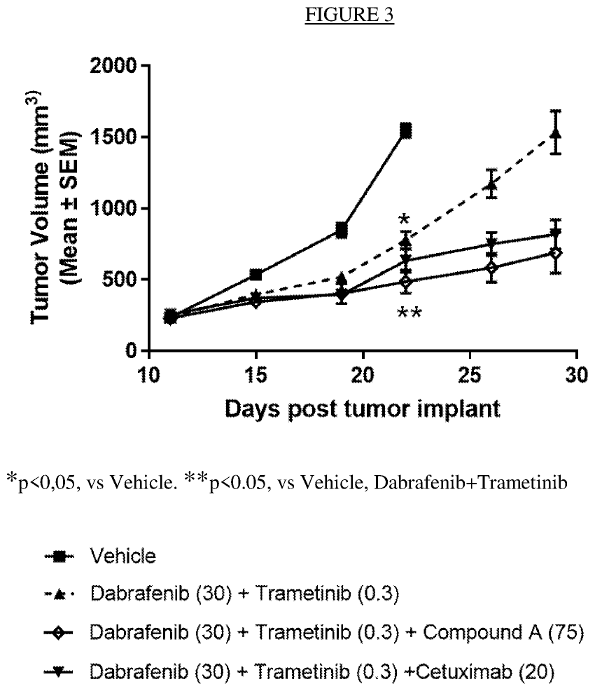

In HCOX5421 model (FIG. 3), the combination of dabrafenib (30 mg/kg, po qd) and trametinib (0.3 mg/kg, po qd) treatment produced a statistically significant anti-tumor effect with 41% T/C. The addition of ERK inhibitor compound A to the dabrafenib+trametinib combination led to further enhanced tumor growth inhibition with 20% T/C, which is statistically significant as compared to the vehicle and combination of dabrafenib+trametinib (p<0.05). In contrast, Cextuximab did not provide significantly improved benefit to the dabrafenib+trametinib combination (29% T/C vs 41% T/C, p>0.05).

These data suggest that the combination of compound A+dabrafenib+trametinib yields improved therapeutic benefit to patients with BRAF V600E CRC relative to the dabrafenib+trametinib double combination.

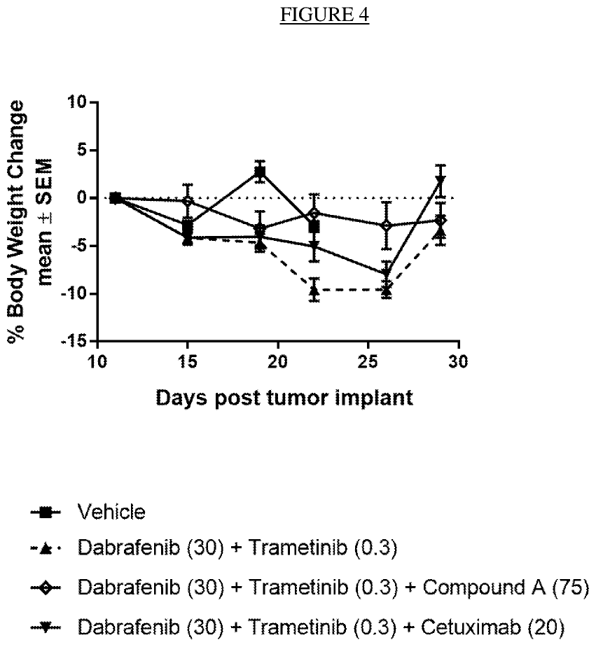

In addition, the mean body weight change for HCOX5421 is shown in FIG. 4. Treatment of mice with a combination of dabrafenib+trametinib, dabrafenib+trametinib+compound A and dabrafenib+trametinib+cetuximab exhibit minimal body weight loss −0.64%, −0.34% and −1.99%, respectively, at the end of treatment, similar to body weight change in the vehicle group. No other signs of adverse events were observed in this study. Almost all animals survived throughout the study except for one in the dabrafenib+trametinib+compound A triple combination group.

### Example 4

In Vivo Antitumor Activity and MAPK Pathway Modulation of Compound A Combined with Dabrafenib and Trametinib in the HT29 BRAF V600E CRC Xenograft Model.

An in vivo antitumor efficacy study, employing female nude mice implanted with the BRAF V600E CRC (colorectal cancer) xenograft model HT29, was conducted to assess the therapeutic benefit of adding the ERK1/2 inhibitor compound A to the combination of the MEK1/2 inhibitor trametinib and the BRAF inhibitor dabrafenib.

HT29 xenografts was established by direct subcutaneous (sc) implantation of 2×10\hat{ }6 cells into the right axillary region of 6-7 week old female nude mice. Mice were randomly assigned to treatment groups (summarized in the table below) 26 days post tumor cell implantation with a tumor volume range between 201 to 611 mm³. dabrafenib was formulated as a solution in 0.5% HPMC+0.2% Tween 80 in pH8 DI water, 3 mg/mL. trametinib was formulated as a solution in 0.5% HPMC, 0.2% Tween80 in pH8 DI Water, 0/03 mg/mL. Compound A was formulated as a suspension in 0.5% HPC/0.5% Pluronic F127 in a pH 7.4 Phosphate Buffer, Final pH adjusted to 4.

| Groups | Treatment | Dose | Schedule | Number of mice |
|---|---|---|---|---|
| 1 | Untreated | N/A | N/A | 9 |
| 2 | compound A | 75 mg/kg | QD, PO | 9 |
| 3 | dabrafenib | 30 mg/kg | QD, PO | 9 |
| 4 | trametinib | 0.3 mg/kg | QD, PO | 9 |
| 5 | dabrafenib + | 30 mg/kg + | QD, PO; | 9 |
| | trametinib | 0.3 mg/kg | QD, PO | |
| 6 | dabrafenib + | 30 mg/kg + | QD, PO; | 9 |
| | trametinib + | 0.3 mg/kg + | QD, PO; | |
| | cetuximab | 20 mg/kg | 2QW, IP | |
| 7 | compound A + | 75 mg/kg + | QD, PO; | 9 |
| | dabrafenib | 30 mg/kg | QD, PO | |
| 8 | compound A + | 75 mg/kg + | QD, PO; | 9 |
| | trametinib | 0.3 mg/kg | QD, PO | |
| 9 | compound A + | 75 mg/kg + | QD, PO; | 9 |
| | dabrafenib + | 30 mg/kg + | QD, PO; | |
| | trametinib | 0.3 mg/kg | QD, PO | |

Animals were weighed at dosing day(s) and dose was body weight adjusted, dosing volume was 10 ml/kg. Tumor dimensions and body weights were collected at the time of randomization and twice weekly thereafter for the study duration.

Percent change in body weight was calculated as (BW_{current}−BWᵢₙᵢₜᵢₐₗ)/(BWᵢₙᵢₜᵢₐₗ)×100. Data is presented as percent body weight change from the day of treatment initiation.

Percent treatment/control (T/C) values were calculated using the following formulae:

% T/C=100×ΔT/ΔC if ΔT>0;

% Regression=100×ΔT/T₀ if ΔT<0;

where:
T=mean tumor volume of the drug-treated group on the final day of the study;
ΔT=mean tumor volume of the drug-treated group on the final day of the study—mean tumor volume of the drug-treated group on initial day of dosing;
T₀=mean tumor volume of the drug-treated group on the day of cohort;
C=mean tumor volume of the control group on the final day of the study; and
ΔC=mean tumor volume of the control group on the final day of the study—mean tumor volume of the control group on initial day of dosing.

All data were expressed as mean±standard error of the mean (SEM). Delta tumor volume and percent body weight changes were used for statistical analysis. Between group comparisons were carried out using the One way ANOVA followed by a post hoc Tukey test. For all statistical evaluations, the level of significance was set at p<0.05. Significance compared to the untreated control group is reported unless otherwise stated.

| | |
|---|---|
| Model | HT29 |
| Treatment | T/C % or Reg % at day 39 post |
| | implantation |
| Untreated | 100 |
| compound A | 53.8 |
| dabrafenib | 58.8 |
| trametinib | 47.7 |
| dabrafenib + trametinib | 28.2 |
| dabrafenib + trametinib + | 4.0 |
| cetuximab | |
| compound A + dabrafenib | 34.6 |
| compound A + trametinib | 9.2 |
| dabrafenib + trametinib + | 2.7 |
| compound A | |

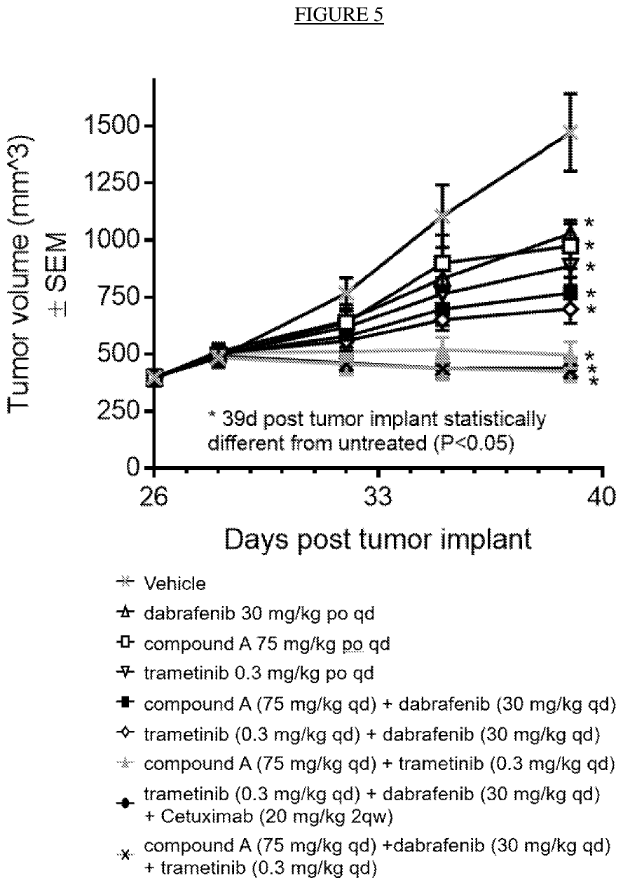

All single agent and combination treatments resulted in in significant tumor growth inhibition relative to untreated control mice (FIG. 5). The greatest antitumor activity was achieved by the following treatments: compound A+trametinib (9.2% T/C), compound A+trametinib+dabrafenib (2.7% T/C), and trametinib+dabrafenib+cetuximab (4.0% T/C).

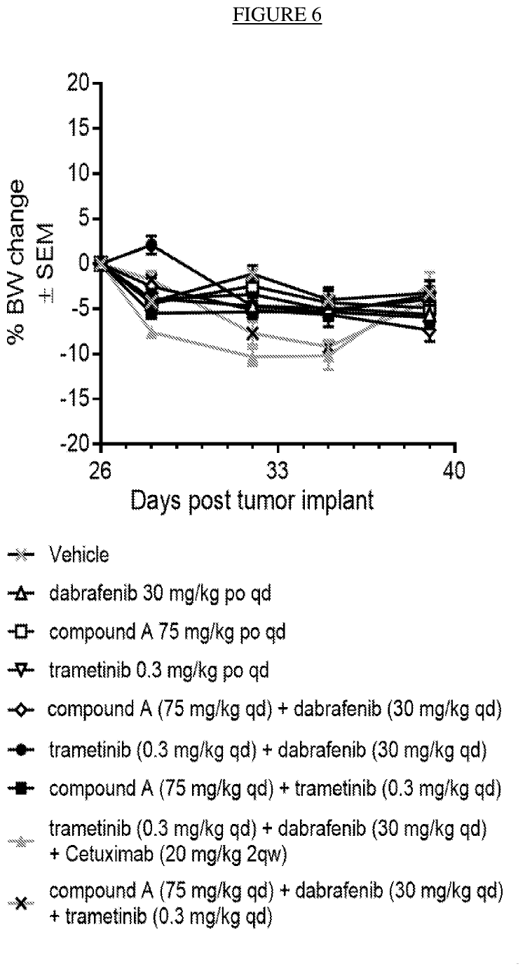

The mean body weight change observed in this study is shown in FIG. 6. At 39 days post tumor implant, all treatment groups experienced a change in BW which was not statistically different from the untreated control group. All mice remained on study throughout the duration of the study.

At the termination of the study following the 14^{th} consecutive daily treatment administration, mice were euthanized at one of three time points post final dose (2, 7, or 24 h). Tumors and dorsal skin were collected and snap frozen in liquid nitrogen for assessment of MAPK pathway output.

Transcriptional read-out of MAPK pathway inhibition was evaluated in tumor and lysates by measuring DUSP6 messenger RNA (mRNA) using quantitative real-time polymerase chain reaction (qPCR). Snap frozen tumor and skin fragments were lysed in RLT buffer (Qiagen, #74104) with the addition of 1% of 2-Mercaptoethanol according to instruction, homogenized using Precellys 24 lysis & a homogenization machine (Bertin Technology). RNA was extracted from snap frozen xenograft fragments using the Qiacube system. Expression of genes (mRNA level) was assessed by one-step PCR using reverse transcription (RT) coupled to real-time quantitative PCR. DUSP6 Taqman Probe (ABI, Hs00737962 ml) was used with QuantiTect Multiplex RT-PCR Kits with ROX dye (Qiagen, 204645) to determine the amount of mRNA expression for DUSP6 relative to the endogenous control gene human RPLPO (Large Ribosomal Protein, HPO, ABI 4326314E) in tumor sample. This one-step PCR method was applied to PD analysis for samples collected, except using probes of mouse DUSP6 (ABI, Mm00518185m1) and beta-actin as endogenous control gene (ABI, 4351315). The PCR reaction was performed on the ABI 7900HT fast real time PCR system. The data were analyzed using the ABI SDS software v2.3 system with an automatic threshold. The difference between the endogenous control gene and the target gene was determined and compared to the calibrator sample (untreated control samples).

Calculation Formulae:

Normalization to endogenous control: ΔCt=Ct target gene−Ct endogenous control.

Normalization to calibrator sample: ΔΔCt=ΔCt sample−ΔCt calibrator.

2^{-(ΔΔCt)} is the value 2 (multiplication of DNA in one cycle) raised to the negative power of the difference in cycle number between the test sample and the calibrator. This value reflects the expression of the test sample relative to the calibrator and is used to calculate the fold difference (RQ value). Once the RQ values have been obtained, the data is further transformed by normalizing the data set to the average RQ values of the vehicle group and calculating the percent mRNA expression of vehicle control using the following formula: % of untreated control=y/k*100.

Where y=RQ value of treatment sample, and k=average RQ value of untreated control.

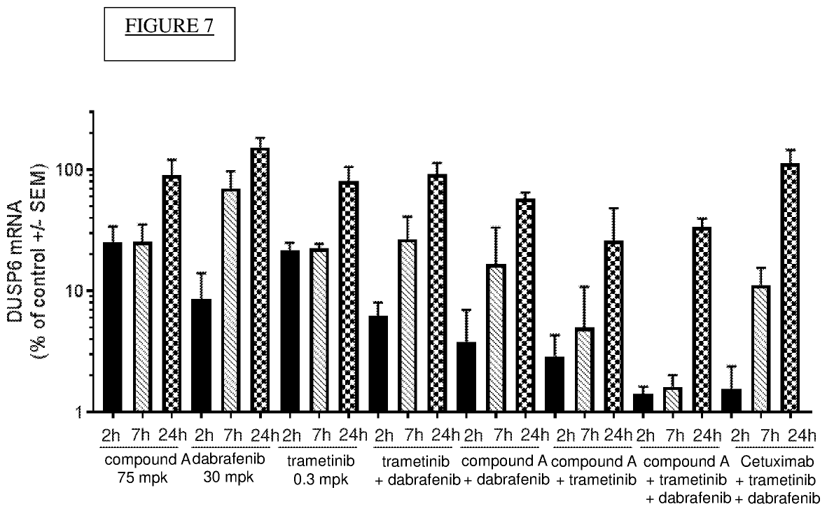

Modulation of MAPK pathway output in tumor and skin was assessed by quantifying transcript abundance of DUSP6 mRNA by qPCR (FIG. 7). The suppression of MAPK pathway output, i.e. DUSP6 mRNA abundance, in tumor was largely consistent with the observed antitumor activity. More specifically, treatment groups that yielded the greatest suppression of tumor DUPS6 mRNA likewise yielded the greatest antitumor activity. For example, compound A+trametinib demonstrated greater pathway suppression and antitumor activity than all single agents as well as the trametinib+dabrafenib and compound A+dabrafenib. Contrary to this, compound A+trametinib, compound A+trametinib+dabrafenib, cetuximab+trametinib+dabrafenib yielded roughly similar antitumor activity in this 14 day experiment despite compound A+trametinib+dabrafenib demonstrating the most comprehensive suppression of MAPK pathway output. A longer term follow-up efficacy study was designed with these treatment groups to test the hypothesis that the compound A+trametinib+dabrafenib combination would yield more durable antitumor response than compound A+trametinib, compound A+trametinib+dabrafenib or cetuximab+trametinib+dabrafenib. This study is summarized in Example 5.

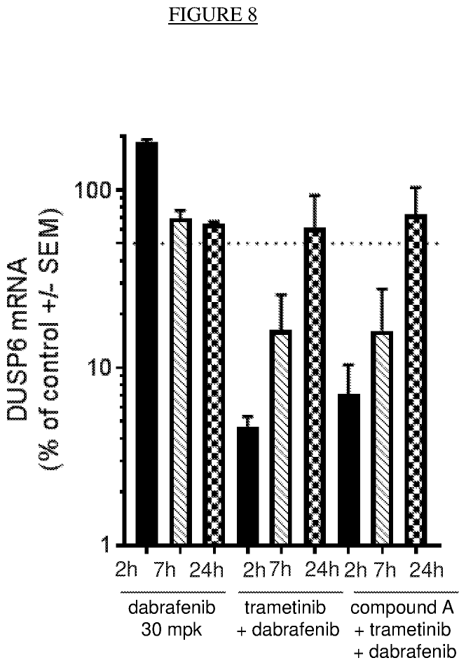

Further, it was observed that addition of dabrafenib to the trametinib+compound A combination resulted in further suppression of MAPK pathway output in the BRAF V600E HT29 xenograft (FIG. 8). In BRAF WT skin, however, the two treatments (trametinib+compound A and dabrafenib+trametinib+compound A) yielded roughly similar suppression of MAPK pathway output (FIG. 6). These data suggest that the addition of dabrafenib could yield improved clinical benefit to BRAF V600E tumors without resulting in increased on-target safety signals associated with MAPK pathway suppression in normal tissues.

### Example 5

In Vivo Antitumor Activity of Compound a Combined with Dabrafenib and Trametinib in the HT29 BRAF V600E CRC xenograft model

An in vivo antitumor efficacy study, employing mice implanted with a BRAF V600E CRC (colorectal cancer) xenograft model HT29, was conducted to assess the durability of therapeutic benefit of associated with adding the ERK1/2 inhibitor compound A to the combination of the MEK1/2 inhibitor trametinib and the BRAF inhibitor dabrafenib.

HT29 xenografts were established by direct subcutaneous (sc) implantation of 2×10\hat{ }6 cells into the right axillary region of 6-7 week old female nude (nu/nu) mice. Mice were randomly assigned to treatment groups (summarized in the table below) 21 days post tumor cell implantation with a tumor volume range between 161.4 to 317.9 mm³. dabrafenib was formulated as a solution in 0.5% HPMC+0.2% Tween 80 in pH8 DI water, 3 mg/mL. trametinib was formulated as a solution in 0.5% HPMC, 0.2% Tween80 in pH8 DI Water, 0/03 mg/mL. Compound A was formulated as suspension in 0.5% HPC/0.5% Pluronic F127 in a pH 7.4 Phosphate Buffer, Final pH adjusted to 4.

| Groups | Treatment | Dose | Schedule | Number of mice |
|---|---|---|---|---|
| 1 | Untreated | N/A | N/A | 7 |
| 2 | dabrafenib + | 30 mg/kg + | QD, PO + | 7 |
| | trametinib | 0.3 mg/kg | QD, PO | |
| 3 | dabrafenib + | 30 mg/kg + | QD, PO + | 7 |
| | trametinib + | 0.15 mg/kg + | QD, PO + | |
| | compound A | 75 mg/kg | QD, PO | |
| 4 | dabrafenib + | 30 mg/kg + | QD, PO; | 7 |
| | trametinib + | 0.3 mg/kg + | QD, PO; | |
| | cetuximab | 20 mg/kg | 2QW, IP | |
| 5 | dabrafenib + | 30 mg/kg + | QD, PO + | 7 |
| | trametinib + | 0.15 mg/kg + | QD, PO + | |
| | compound A | 75 mg/kg | QD, PO | |

Animals were weighed at dosing day and dose was body weight adjusted, dosing volume was 10 ml/kg. Tumor dimensions and body weights were collected at the time of randomization and twice weekly thereafter for the study duration.

Percent change in body weight was calculated as (BW_{current}−BWᵢₙᵢₜᵢₐₗ)/(BWᵢₙᵢₜᵢₐₗ)×100. Data is presented as percent body weight change from the day of treatment initiation.

Percent treatment/control (T/C) values were calculated using the following formulae:

% T/C=100×ΔT/ΔC if ΔT>0%

Regression=100×ΔT/T₀ if ΔT<0

where:

T=mean tumor volume of the drug-treated group on the final day of the study;

ΔT=mean tumor volume of the drug-treated group on the final day of the study—mean tumor volume of the drug-treated group on initial day of dosing;

T₀=mean tumor volume of the drug-treated group on the day of cohort;

C=mean tumor volume of the control group on the final day of the study; and

ΔC=mean tumor volume of the control group on the final day of the study—mean tumor volume of the control group on initial day of dosing.

All data were expressed as mean±standard error of the mean (SEM). Delta tumor volume and percent body weight changes were used for statistical analysis. Between group comparisons were carried out using the One way ANOVA followed by a post hoc Tukey test. For all statistical evaluations, the level of significance was set at p<0.05. Significance compared to the untreated control group is reported unless otherwise stated.

| | |
|---|---|
| Model | HT29 |
| Treatment | T/C % or Reg % at day 42 post |
| | implantation |
| Untreated | 100 |
| dabrafenib + trametinib | 52.3 |
| compound A + trametinib | 19.8 |
| cetuximab + dabrafenib + | 11.0 |
| trametinib | |
| compound A + dabrafenib + | 6.5 |
| trametinib | |

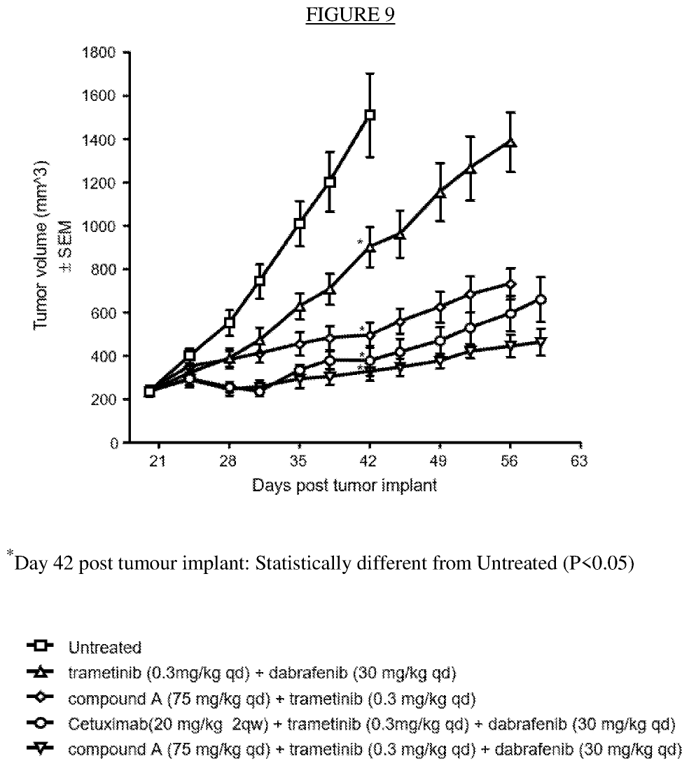

This second study with mice bearing HT29 BRAF V600E CRC xenografts was conducted to follow up on observations made in Example 4. Specifically, this second experiment was designed to incorporate longer term dosing with the goal of determining if the more comprehensive MAPK pathway suppression achieved in HT29 xenografts with the dabrafenib+trametinib+compound A combination (relative to trametinib+compound A and cetuximab+dabrafenib+trametinib) translated into more durable tumor growth control. All treatment groups yielded significant tumor growth inhibition relative to untreated control mice at 42 days post tumor cell implantation (FIG. 9). With continued dosing, the compound A+dabrafenib+trametinib combination yielded numerically greater antitumor activity, suggesting improved durability of therapeutic benefit, than all other treatments including cetuximab+dabrafenib+trametinib.

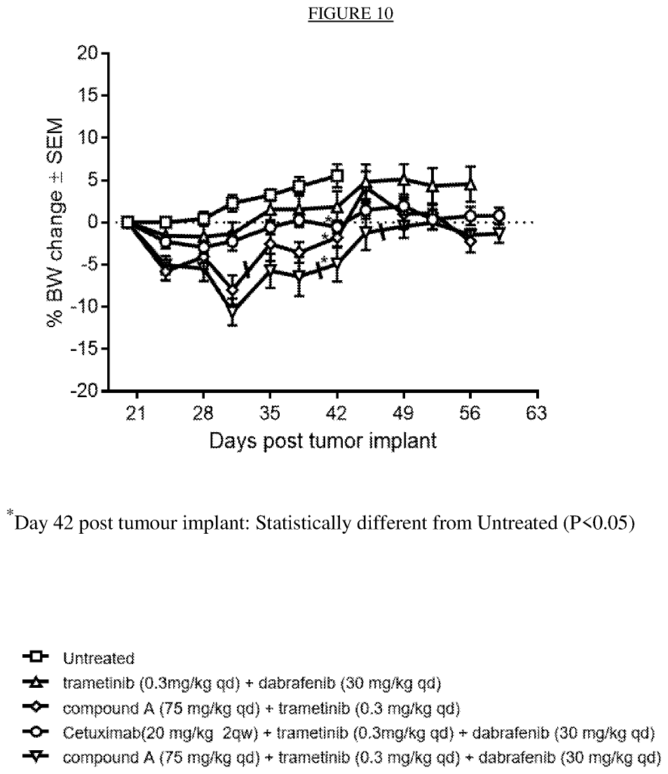

The mean body weight change observed in this study is shown in FIG. 10. At 42 days post tumor implant, all treatment groups with the exception of trametinib+dabrafenib, experienced a change in BW that was lower than the untreated control group (P<0.05). Hash marks indicate days in which a mouse was removed from treatment early. This included two mice from the dabrafenib+trametinib+compound A group and one from the compound A+trametinib group.

It is understood that the Examples and embodiments described herein are for illustrative purposes only and that various modifications or changes in light thereof will be suggested to persons skilled in the art and are to be included within the spirit and purview of this application and scope of the appended claims.

1. A pharmaceutical combination comprising N-(3-(5-(2-aminopyrimidin-4-yl)-2-(tert-butyl)thiazol-4-yl)-2-fluorophenyl)-2,6-difluorobenzenesulfonamide (dabrafenib), or a pharmaceutically acceptable salt thereof, N-(3-(3-cyclopropyl-5-((2-fluoro-4-iodophenyl)amino)-6,8-dimethyl-2,4,7-trioxo-3,4,6,7-tetrahydropyrido[4,3-d]pyrimidin-1(2H)-yl)phenyl)acetamide (trametinib), or a pharmaceutically acceptable salt or solvate thereof, and 4-(3-amino-6-((1 S,3 S,4 S)-3-fluoro-4-hydroxycyclohexyl)pyrazin-2-yl)-N—((S)-1-(3-bromo-5-fluorophenyl)-2-(methylamino)ethyl)-2-fluorobenzamide (compound A), or a pharmaceutically acceptable salt thereof.

2. The combination of claim 1, wherein N-(3-(5-(2-aminopyrimidin-4-yl)-2-(tert-butyl)thiazol-4-yl)-2-fluorophenyl)-2,6-difluorobenzenesulfonamide (dabrafenib), or a pharmaceutically acceptable salt thereof, N-(3-(3-cyclopropyl-5-((2-fluoro-4-iodophenyl)amino)-6,8-dimethyl-2,4,7-trioxo-3,4,6,7-tetrahydropyrido[4,3-d]pyrimidin-1(2H)-yl)phenyl)acetamide (trametinib), or a pharmaceutically acceptable salt thereof, and 4-(3-amino-6-((1 S,3 S,4S)-3-fluoro-4-hydroxycyclohexyl)pyrazin-2-yl)-N—((S)-1-(3-bromo-5-fluorophenyl)-2-(methylamino)ethyl)-2-fluorobenzamide (compound A), or a pharmaceutically acceptable salt thereof, are administered separately, simultaneously or sequentially, in any order.

3. The pharmaceutical combination according to claim 1, which is for oral administration.

4. The pharmaceutical combination according to claim 1, wherein N-(3-(5-(2-aminopyrimidin-4-yl)-2-(tert-butyl)thiazol-4-yl)-2-fluorophenyl)-2,6-difluorobenzenesulfonamide (dabrafenib) is in an oral dosage form.

5. The pharmaceutical combination of claim 1, wherein N-(3-(3-cyclopropyl-5-((2-fluoro-4-iodophenyl)amino)-6,8-dimethyl-2,4,7-trioxo-3,4,6,7-tetrahydropyrido[4,3-d]pyrimidin-1(2H)-yl)phenyl)acetamide (trametinib) is in an oral dosage form.

6. The pharmaceutical combination according to claim 1, wherein 4-(3-amino-6-((1S,3 S,4S)-3-fluoro-4-hydroxycyclohexyl)pyrazin-2-yl)-N—((S)-1-(3-bromo-5-fluorophenyl)-2-(methylamino)ethyl)-2-fluorobenzamide (compound A) is in an oral dosage form.

7. A pharmaceutical composition or a commercial package comprising the pharmaceutical combination according to of claim 1 and at least one pharmaceutically acceptable carrier.

8. A method of treating cancer, to a patient in need thereof, by administering a pharmaceutical combination according to claim 1 or the pharmaceutical composition or the commercial package according to claim 7.

9. The method of claim 8, wherein the cancer is selected from breast cancer, cholangiocarcinoma, colorectal cancer (CRC), melanoma, non-small cell lung cancer, ovarian cancer and thyroid cancer.

10. The method of claim 8, wherein the cancer is advanced or metastatic colorectal cancer, BRAF gain of function CRC or BRAF V600E CRC.

11. (canceled)

12. (canceled)

13. (canceled)

14. The method of claim 8, wherein N-(3-(5-(2-aminopyrimidin-4-yl)-2-(tert-butyl)thiazol-4-yl)-2-fluorophenyl)-2,6-difluorobenzenesulfonamide (dabrafenib) is administered orally at a dose of about from about 1, 2, 5, 10, 50, 100 or to about 150 mg per day.

15. The method of claim 14, wherein N-(3-(3-cyclopropyl-5-((2-fluoro-4-iodophenyl)amino)-6,8-dimethyl-2,4,7-trioxo-3,4,6,7-tetrahydropyrido[4,3-d]pyrimidin-1(2H)-yl)phenyl)acetamide (trametinib) is administered orally at a dose of from about 0.5 to, 1, or 2 mg per day and, about 0.5635, 1.127 or 2.254 mg per day in the form of trametinib dimethyl sulfoxide).

16. The method of claim 15, wherein 4-(3-amino-6-((1S,3S,4S)-3-fluoro-4-hydroxycyclohexyl)pyrazin-2-yl)-N—((S)-1-(3-bromo-5-fluorophenyl)-2-(methylamino)ethyl)-2-fluorobenzamide (compound A) is administered orally at a dose of from about 50, 75 to about 100 mg per day.
